(19) United States
(12) Patent Application Publication
(10) Pub. No.: US 2017/0173212 A1
(43) Pub. Date: Jun. 22, 2017

(54) COMPOSITIONS AND METHODS FOR GROWING AUTOLOGOUS BIOLOGICAL TISSUE

(71) Applicant: BOARD OF REGENTS, THE UNIVERSITY OF TEXAS SYSTEM, AUSTIN, TX (US)

(72) Inventor: Liping TANG, Arlington, TX (US)

(21) Appl. No.: 15/129,126

(22) PCT Filed: Mar. 26, 2015
(86) PCT No.: PCT/US2015/022680

§ 371 Date: Sep. 26, 2016

Related U.S. Application Data
(60) Provisional application No. 61/971,351, filed on Mar. 27, 2014.

(51) Int. Cl. A61L 27/22; A61L 27/24; C08L 89/06; A61L 27/54; C08L 5/08; A61L 27/56; A61L 27/18

(52) U.S. Cl. CPC A61L 27/222; A61L 27/56; A61L 27/24; A61L 27/18; A61L 27/54; C08L 5/08; C08L 89/06; A61L 2430/20; A61L 2430/02; A61L 2430/34; A61L 2300/254; A61L 2300/412

(57) ABSTRACT

In one aspect, methods of growing autologous biological tissue are described herein. In some embodiments, a method described herein comprises disposing a porous scaffold in a body cavity of a patient, wherein the scaffold comprises one or more biofactors comprising one or more chemokines that promote migration of autologous multipotent cells into the body cavity and/or one or more differentiation agents that promote differentiation of autologous multipotent cells into the autologous biological tissue. The body cavity can comprise a soft tissue cavity such as the peritoneal cavity. A method described herein can also comprise depositing the multipotent cells onto a surface of the scaffold and inducing differentiation of the multipotent cells on the surface of the scaffold to provide differentiated autologous tissue. Additionally, a method can further comprise growing the autologous biological tissue from the differentiated autologous tissue. Further, the autologous tissue may not be native to the body cavity.

### CROSS REFERENCE TO RELATED APPLICATIONS

This application claims priority pursuant to 35 U.S.C. §119 to U.S. Provisional Patent Application Ser. No. 61/971,351, filed on Mar. 27, 2014, which is hereby incorporated by reference in its entirety.

### STATEMENT REGARDING FEDERALLY SPONSORED RESEARCH

This invention was made with government support under grant number EB007271, awarded by the National Institutes of Health. The Government has certain rights in the invention.

### FIELD

This invention relates to compositions and methods for growing tissue and, in particular, to compositions and methods for growing autologous biological tissue such as bone in vivo.

### BACKGROUND

Bone loss can occur in a variety of ways. For example, bone loss often occurs as a result of open fractures, osteomyelitis, fractures which fail to heal, congenital malformations, tumors, and in a more general sense, osteoporosis. In recent years, significant progress has been made in the development of materials designed to replace or bridge large bone defects. For instance, some strategies involve the use of an implant, such as a three-dimensional scaffold, that integrates with existing bone tissue to restore bone and/or replace the function of damaged bone. Other strategies involve the use of ex vivo tissue growth. However, these approaches can be limited by contamination of external materials, the survival or “shelf life” of ex vivo tissue, and the need to carry out extensive harvesting and purification protocols. Therefore, there remains a need for improved methods and materials for promoting bone growth and/or repair. Similarly, there remains a need for improved methods and materials for growing other biological tissues as well as bone, such as cartilage tissue, tendon tissue, fat tissue, nerve tissue, heart valve tissue, kidney tissue, pancreas tissue, and liver tissue.

### SUMMARY

In one aspect, methods of growing autologous biological tissue are described herein which, in some embodiments, may provide one or more advantages compared to some other methods. For example, in some instances, a method described herein can permit a patient to grow his or her own differentiated or specialized tissue, including for subsequent transplantation to a different portion of the patient's body than the location in which the differentiated or specialized tissue was grown. Moreover, such growth of autologous tissue can be achieved without one or more disadvantages of some ex vivo methods. For instance, in some ex vivo methods, desired cells must be harvested from the patient's body and possibly purified. The harvested cells must then be further cultured and/or grown outside of the patient's body, such as by providing the cells with a regimen of nutrients and/or placing the cells in an external bioreactor. Further, once the desired tissue is grown from the harvested cells outside the patient's body, the tissue must then be reintroduced into the patient, which can lead to contamination and/or the introduction of foreign substances. Such reintroduction may also result in rejection of the implanted tissue by the patient. In addition, tissue grown in ex vivo can have a limited “shelf life” or period of time in which the tissue must or should desirably be implanted in the patient following tissue growth. In contrast to such ex vivo methods, some methods described herein can be carried out without undertaking one or more of the foregoing steps and/or without suffering from one or more of the foregoing disadvantages. More generally, methods described herein can be used to grow autologous tissues in a manner that is safer and/or simpler than some other methods.

In addition, methods described herein can also provide autologous tissue-containing scaffolds or implants that can be transplanted to another portion of a patient's body (other than where the autologous tissue was grown in or on the scaffold or implant) without the need to first separate the autologous tissue from the scaffold or implant. Instead, methods described herein, in some cases, can comprise transplanting an autologous tissue-containing scaffold or implant to another portion of a patient's body (the transplant location) and then biodegrading the scaffold or implant in vivo in the transplant location, including in proportion to the continued growth of the autologous tissue in the transplant location.

In some embodiments, a method of growing autologous biological tissue described herein comprises disposing a scaffold or implant in a desired region of a living patient, wherein the scaffold or implant comprises one or more biofactors. The biofactors comprise one or more chemokines that promote migration of autologous multipotent cells into or toward the desired region and/or one or more differentiation agents that promote differentiation of autologous multipotent cells, including into the autologous biological tissue. In some cases, the biofactors comprise a combination of such chemokines and differentiation agents. Additionally, in some embodiments, a method described herein further comprises releasing at least a portion of the biofactors into the region from the scaffold or implant.

In some instances, the region in which the scaffold or implant is disposed is a body cavity of the patient. Moreover, such a body cavity can comprise a soft tissue cavity such as the peritoneal cavity, the pleural cavity, or the abdominal cavity. The multipotent cells may be stem cells such as mesenchymal stem cells (MSCs) or progenitor cells. Further, in some cases, the multipotent cells are native to the body cavity (or other desired region). In other instances, the multipotent cells are not native to the body cavity (or other desired region).

In addition, in some embodiments, a method described herein further comprises depositing the autologous multipotent cells onto a surface of the scaffold, including in response to a biological signal provided by one or more biofactors of a scaffold described herein. The surface can be an interior or exterior surface of the scaffold, such that the cells are deposited “in” or “on” the scaffold. A method described herein can also comprise inducing differentiation of the autologous multipotent cells on the interior and/or exterior surface of the scaffold to provide differentiated autologous tissue. As described further hereinbelow, the differentiation can be induced by the biofactors of the scaffold. Moreover, in some embodiments, a method described herein further comprises growing the autologous biological tissue from the differentiated autologous tissue on the surface of the scaffold. Again, it is to be understood that such growth of differentiated or specialized autologous tissue can occur on an exterior surface of the scaffold and/or within the interior of the scaffold. Further, as described above, the autologous biological tissue may not be native to the body cavity (or other region) in which the scaffold is disposed and in which growth of the tissue occurs. Thus, in some instances, a method described herein can permit one portion of a patient's body to serve as an internal or in vivo bioreactor for the growth of new autologous tissue of the patient, including for eventual transplantation to a different region or portion of the patient's body for therapeutic purposes. In some embodiments, therefore, a method described herein further comprises removing the autologous biological tissue (including while still attached to or disposed within the scaffold) from the body cavity (or other region) of the patient and implanting the autologous biological tissue in a portion of the patient that differs from the body cavity. In this manner, many of the disadvantages of some ex vivo methods may be reduced or completely eliminated.

In another aspect, compositions are described herein. In some embodiments, a composition described herein comprises a scaffold or implant and one or more biofactors disposed in the scaffold or implant. The scaffold or implant, in some cases, is a porous scaffold or implant, such as a scaffold or implant having an average pore size of 50-250 μm. Further, the scaffold or implant can be biodegradable and/or biocompatible. In addition, in some instances, the biofactors disposed in the scaffold or implant comprise one or more chemokines that can promote migration of autologous multipotent cells to a surface and/or interior region of the scaffold or implant, and/or one or more differentation agents that can promote differentiation of autologous multipotent cells into autologous biological tissue, including at a site in which the scaffold or implant is initially disposed. Moreover, in some embodiments, the amount of the biomarkers disposed in the scaffold is an amount that selectively promotes tissue growth on or in the scaffold or implant, as opposed to tissue growth that may occur elsewhere, including elsewhere within a body cavity (or other region) in which the scaffold or implant is disposed.

Compositions described herein, in some cases, can thus provide one or more advantages compared to some prior compositions, including for tissue growth applications. For instance, in some embodiments, a composition described herein can be used to carry out a method of growing autologous biological tissue in a body cavity (or other region), as described hereinabove. In addition, in some instances, a composition described herein can exhibit a long shelf life or period of stability after fabrication of the composition, such that the “ready made” scaffold or implant of the composition can be inserted into a patient at any desired time to carry out a tissue growth method described herein. A composition described herein can also exhibit or induce no rejection response or only a minimal rejection response from the patient. A composition described herein can also reduce the risk of contamination and/or the introduction of harmful foreign substances into a patient during a tissue growth, transplant, or other therapeutic procedure.

These and other embodiments are described in more detail in the detailed description which follows.

### BRIEF DESCRIPTION OF THE FIGURES

FIG. 1 illustrates a graph of results associated with methods of growing autologous biological tissue according to some embodiments described herein.

FIG. 2 illustrates a graph of results associated with methods of growing autologous biological tissue according to some embodiments described herein.

FIG. 3 illustrates a graph of results associated with methods of growing autologous biological tissue according to some embodiments described herein.

FIG. 4 illustrates a graph of results associated with methods of growing autologous biological tissue according to some embodiments described herein.

FIG. 5 illustrates a graph of results associated with methods of growing autologous biological tissue according to some embodiments described herein.

FIG. 6 illustrates a graph of results associated with methods of growing autologous biological tissue according to some embodiments described herein.

FIG. 7 illustrates a graph of results associated with methods of growing autologous biological tissue according to some embodiments described herein.

FIG. 8 illustrates a graph of results associated with methods of growing autologous biological tissue according to some embodiments described herein.

FIG. 9 illustrates an optical micrograph of a scaffold of a composition according to one embodiment described herein.

FIG. 10 illustrates an optical micrograph of a scaffold of a composition according to one embodiment described herein.

### DETAILED DESCRIPTION

Embodiments described herein can be understood more readily by reference to the following detailed description, examples, and figures. Elements, apparatus, and methods described herein, however, are not limited to the specific embodiments presented in the detailed description, examples, and figures. It should be recognized that these embodiments are merely illustrative of the principles of the present invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” should generally be considered to include the end points 5 and 10.

### I. Methods of Growing Autologous Biological Tissue

In one aspect, methods of growing autologous biological tissue are described herein. In general, such methods can comprise disposing a scaffold or implant in a desired region, site, or biological compartment of a patient, and then inducing and/or promoting autologous tissue growth in the region, site, or compartment. In some cases, inducing and/or promoting autologous tissue growth comprises “recruiting” autologous multipotent cells from the patient and/or inducing differentiation of such autologous multipotent cells into the autologous tissue, including in the desired region, site, or biological compartment. “Recruiting” cells, as described herein, can refer to attracting cells or otherwise incorporating the cells into a step of a method described herein. Additionally, the “recruitment” and/or differentiation of cells, in some instances, can be achieved through the use of one or more biofactors disposed in or on the scaffold. For example, in some embodiments, the biofactors can comprise one or more chemokines that promote migration of autologous multipotent cells into the region, site, or compartment and/or to a surface of the scaffold. The biofactors may also comprise one or more differentiation agents that promote differentiation of autologous multipotent cells into the autologous biological tissue. In some cases, the biofactors comprise a combination of one or more chemokines and one or more differentiation agents. Moreover, in some embodiments, a single biofactor may serve to promote migration as well as differentiation of autologous multipotent cells in a manner described herein. In other words, such a single biofactor may be both a chemokine and a differentiation agent. In other instances, a specific biofactor provides only one of the two effects.

For example, in some embodiments, a method of growing autologous biological tissue comprises disposing a scaffold in a body cavity of a living patient, wherein the scaffold comprises one or more biofactors. The biofactors can comprise one or more chemokines that promote migration of autologous multipotent cells into the body cavity and/or to a surface of the scaffold, including an interior surface of the scaffold, as opposed to or in addition to an exterior surface of the scaffold. The biofactors may also comprise one or more differentiation agents that promote differentiation of autologous multipotent cells into the autologous biological tissue. In some cases, the biofactors comprise a combination of one or more chemokines and one or more differentiation agents.

Additionally, in some cases, a method described herein further comprises releasing at least a portion of the biofactors into the body cavity from the scaffold. In some instances, all or substantially all of the biofactors are released into the body cavity from the scaffold, where “substantially” all of the biofactors can comprise at least about 70%, at least about 80%, at least about 90%, or at least about 95% of the total amount of the biofactors. Moreover, as described further hereinbelow, biofactors can be released from the scaffold over a sustained period of time, such as a period of 1-10 weeks. Further, in some instances, less than about 30%, less than about 20%, or less than about 10% of the biofactors are released during the first week following placement of the scaffold in the body cavity. In some cases, releasing biofactors in this manner can provide or create a localized gradient of biofactors proximate the scaffold and/or within the body cavity. The localized gradient, in some instances, can guide cell migration, as described further herein. Moreover, in some embodiments, such a release of biofactors can be achieved by disposing the biofactors on or in a porous scaffold described herein, including in an amount or concentration described herein. The use of a porous scaffold described herein can also permit the deposition of cells and/or the growth of tissue within the scaffold, as opposed to only on the exterior surface of the scaffold. As described further herein, such a scaffold including autologous tissue within the scaffold can, in some instances, provide one or more advantages over a scaffold having tissue only on its exterior surface. For example, in some embodiments, a scaffold including autologous tissue within the scaffold can permit safer, simpler, and/or less expensive transplantation of the autologous tissue to another region of a patient's body, including for purposes of treating a defect or disease site within the patient.

The multipotent cells of a method described herein, in some cases, are native to the body cavity (or other region). In other instances, the multipotent cells are not native to the body cavity (or other region). Cells that are “native” to a body cavity (or other region), for reference purposes herein, comprise cells that are ordinarily found in the body cavity (or other region) of the patient in a detectable amount or an amount above a minimum threshold amount. Similarly, cells that are “not native” to a body cavity (or other region), for reference purposes herein, comprise cells that are not ordinarily found in the body cavity (or other region) in a detectable amount or an amount above a minimum threshold, such as an amount of about 0.05%, 0.5%, about 1%, or about 5%, based on the total number of cells. In addition, it is to be understood that, in some embodiments of methods described herein, disposing a scaffold in a body cavity (or other region) can result in the recruitment of multipotent cells that are already present in or native to the body cavity (or other region) as well as recruitment of multipotent cells that are not initially present in or not native to the body cavity (or other region) but subsequently migrate to the body cavity (or other region) from other areas of the patient's body.

A method described herein, in some cases, further comprises depositing the autologous multipotent cells onto a surface of the scaffold disposed in the body cavity (or other region). Moreover, such a method can also comprise inducing differentiation of the autologous multipotent cells on the surface of the scaffold to provide differentiated autologous cells or differentiated autologous tissue. As described further herein, the differentiation can be induced by one or more of the biofactors of the scaffold, such as one or more differentiation agents. Additionally, in some embodiments, a method described herein further comprises growing the autologous biological tissue from the differentiated autologous cells or tissue on the surface of the scaffold.

Further, in some cases, the autologous biological tissue that is grown by a method described herein is not native to the body cavity (or other region) in which the growth is carried out. Autologous tissue that is “not native” to a body cavity (or other region), for reference purposes herein, comprises autologous tissue that does not ordinarily grow in the body cavity (or other region) and/or is not ordinarily found in the body cavity (or other region) in a healthy patient. For example, bone tissue is not native to the peritoneal cavity in humans, and heart valve tissue is not native to the pleural cavity. Therefore, as described further herein, a method according to the present disclosure can be used to grow autologous biological tissue within a patient in a site other than a region in which such tissue ordinarily grows. Moreover, such autologous tissue can subsequently be removed from the body cavity or other region (while still associated with or connected to the scaffold or not) and transplanted into a different biological compartment of the patient, such as a compartment comprising a therapeutic or treatment site or a defect site. Moreover, such a site may be a site where the autologous tissue ordinarily does grow. In some instances, therefore, a method described herein further comprises removing the autologous biological tissue from the body cavity (or other region) of the patient and implanting the autologous biological tissue in a portion of the patient that differs from the body cavity (or other region) of the patient.

Thus, in another aspect, therapeutic methods are described herein. Such methods can include methods of treating a bone defect, a heart defect, or another defect. Therapeutic methods described herein can also include methods of treating lost or diseased tissue, including by growing replacement biological tissue, wherein the replacement biological tissue can comprise any autologous biological tissue grown by a method described herein. Such therapeutic methods can thus comprise the steps of growing autologous biological tissue in a manner described herein and disposing the autologous biological tissue in a defect site or other treatment site. In some embodiments, the defect site or other treatment site can comprise a site within the patient comprising damaged, diseased, malfunctioning, or missing tissue. For example, in some cases, a defect site can comprise a bone defect site where bone tissue is missing or absent. In other instances, a defect site can comprise a heart defect site where heart tissue is damaged or malfunctioning, such as a defective heart valve site.

Moreover, a therapeutic method described herein can further comprise removing damaged or malfunctioning tissue from the defect site, either before, during, or after disposing the replacement autologous biological tissue in the defect site or other treatment site. Further, as described above, the replacement autologous biological tissue can be associated with, attached to, and/or disposed within the scaffold on which the replacement autologous biological tissue was grown in a manner described hereinabove. It is also possible for the replacement autologous biological tissue to be provided to the defect or other treatment site without the scaffold or following removal of the scaffold from the autologous biological tissue. Additionally, in some cases, the scaffold is biodegradable, and the scaffold subsequently degrades and dissipates from the defect site, including in proportion to the continued growth of the replacement biological tissue, in proportion to the integration of the replacement biological tissue into the defect site or other treatment site, and/or in proportion to the integration of the replacement biological tissue with other biological tissue in contact with or adjacent to the defect site or other treatment site. Replacement biological tissue grown and/or used therapeutically in a manner described herein can include any autologous biological tissue described herein. For example, in some instances, the replacement biological tissue comprises a replacement heart valve, replacement vasculature, or replacement diaphragm membrane. Other replacement tissues may also be grown and used in a manner described herein.

Turning now more particularly to specific steps of methods described herein, methods described herein comprise disposing a scaffold or implant in a body cavity or other region of a patient. The patient can be a living human or animal patient. In addition, the scaffold or implant can comprise any scaffold or implant not inconsistent with the objectives of the present disclosure. Moreover, a “scaffold” can refer to any structure usable as a platform or implant for the growth of new tissue. Further, the terms “scaffold” and “implant” can be used interchangeably herein. In some embodiments, a scaffold described herein comprises or is formed from a synthetic or non-naturally occurring polymer or oligomer. In other instances, a scaffold comprises or is formed from a naturally occurring polymer or oligomer. In some cases, for example, a scaffold comprises, consists, or consists essentially of a polylactide (PLA) such as a poly-D,L-lactide, poly-D-lactide, or poly-L-lactide. A scaffold can also comprise, consist, or consist essentially of a polyglycolide, a polycaprolactone (PCL) such as poly-ε-caprolactone, or a polyhydroxyalkanoate (PHA). In some embodiments, a scaffold comprises a mixture or copolymer of one or more of the foregoing. In some cases, a scaffold comprises or is formed from one or more of a poly-L-lactic acid (PLLA), poly-L-glycolic acid (PLGA), PLLA-PLGA copolymer or polymer blend, polycaprolactone, polydioxanone, poly-3-hydroxybutyrate, and polytartronic acid. In some embodiments, a scaffold comprises or is formed from one or more of collagen, hyaluronic acid, and gelatin. Additionally, in some instances, a scaffold described herein can comprise or be formed from decalcified bone or teeth, or from fragments and/or powders of bone or teeth. In other cases, a scaffold does not comprise or is not formed from decalcified bone or teeth, or from fragments and/or powders of bone or teeth. Other materials may also be used to form a scaffold described herein.

Further, in some cases, a scaffold described herein is biocompatible or formed from one or more biocompatible materials, including materials described hereinabove. More particularly, a biocompatible scaffold, in some embodimetns, is non-toxic and does not cause substantial tissue inflammation and/or an immune response from the patient.

A scaffold described herein can also be biodegradable or formed from one or more biodegradable materials. In some instances, a biodegradable scaffold degrades in vivo to non-toxic components which can be cleared from the body by ordinary biological processes. Such processes can include biologically assisted mechanisms, such as enzyme catalyzed reactions, or chemical mechanisms, such as hydrolysis. Moreover, in some embodiments, a biodegradable scaffold described herein completely or substantially completely degrades in vivo over the course of about 90 days or less, about 60 days or less, about 30 days or less, or about 15 days or less, where the extent of degradation is based on percent mass loss of the scaffold, and wherein complete degradation corresponds to 100% mass loss. Specifically, the mass loss is calculated by comparing the initial weight (W₀) of the scaffold with the weight measured at a pre-determined time point (Wₜ) (such as 60 days), as shown in equation (1):

$$Mass loss ( \% ) = \frac{( {W}_{0} - {W}_{t} )}{{W}_{0}} \times 100. \qquad ( 1 )$$

In some cases, a biodegradable scaffold described herein completely or substantially completely degrades in vivo over the course of about 15-120 days, about 30-120 days, 30-90 days, or 30-60 days.

Moreover, in some embodiments, a scaffold described herein is a porous scaffold. In some cases, a porous scaffold described herein has a porosity between about 10% and about 99%, between about 30% and about 90%, between about 30% and about 85%, between about 30% and about 80%, between about 30% and about 70%, between about 50% and about 90%, or between about 50% and about 80%, based on the total volume of the scaffold. The porosity of a scaffold can be measured in any manner not inconsistent with the objectives of the present disclosure. In some embodiments, for instance, porosity is measured by determining the bulk volume of the porous scaffold and subtracting the volume of the material from which the scaffold is formed. Other methods may also be used. Additionally, a porous scaffold can exhibit any range of pore sizes not inconsistent with the objectives of the present disclosure. In some cases, for instance, a scaffold exhibits an average pore size of about 1-1000 μm, about 1-500 μm, about 10-1000 μm, about 10-500 μm, about 10-300 μm, about 30-1000 μm, about 30-500 μm, about 50-1000 μm, about 50-500 μm, or about 50-250 μm.

Scaffolds described herein also comprise one or more biofactors. As understood by one of ordinary skill in the art, a “biofactor” or “biological factor” can be any substance that produces a biological effect in an organism. Moreover, the biofactors of a scaffold described herein can comprise one or more chemokines and/or one or more differentiation agents. A chemokine, as described herein, can promote, facilitate, or cause migration of another biological species within an organism. For instance, a chemokine can promote or facilitate the migration or movement of a cell of a specific type to or from a specific location within an organism, such as a body cavity described herein. Further, a chemokine can promote or facilitate such migration or movement in any manner not inconsistent with the objectives of the present disclosure. For instance, a chemokine can provide a chemical or biological signal or “cue” to a specific biological species, such as a cell of a specific type.

Similarly, a differentiation agent, as described herein, can promote, facilitate, or cause a cell to undergo differentiation. Moreover, the differentiation promoted, facilitated, or caused by a differentiation agent can comprise one or more specific differentiation steps for a given cell. For instance, in some cases, a differentiation agent promotes, facilitates, or causes osteogenic differentiation. Other types of differentiation are also contemplated herein. Further, as understood by one of ordinary skill in the art, the effect of a chemokine and/or a differentiation agent, in some cases, can be specific to a given cell type and/or to a desired autologous biological tissue to be grown from a multipotent cell.

In addition, in some embodiments, at least one chemokine and at least one differentiation agent of a scaffold can promote movement and differentiation, respectively, of the same type of autologous multipotent cell. Thus, in some cases wherein a scaffold comprises at least one chemokine and also at least one differentiation agent, the chemokine and the differentiation agent can be a chemokine and a differetiation agent for the same type of cell, such as the same type of progenitor cell. In this manner, a scaffold described herein can be operable to both “recruit” and cause the differentiation of multipotent cells within a body cavity.

Non-limiting examples of chemokines suitable for use in some embodiments described herein include bioactive lipids such as Sphingosine-1-phosphate (S1P) and ceramide-1-phosphate (C1P); chemokine ligands such as CCL2, CCL3, CCL5, CCL7, CCL 19-22, CCL25, CCL28, CXCL8, CXCL10-13, CXCL16 and CX₃CL1; cationic antimicrobial peptides (CAMPs) such as LL-37, C1q, and C3a; and matrix metalloproteinases (MMPs) such as MMP-9. Additional non-limiting examples of chemokines suitable for use in some embodiments described herein include Erythropoietin (Epo); Granulocyte-colony stimulating factor (GCSF); Hepatocye growth factor (HGF); Interleukin-1 beta (IL-1β); Interleukin-8 (IL-8); Monocyte Chemotactic Protein-1 (MCP-1); Regulated on activation normally T-cell expressed and secreted (RANTES); Stem cell factor (SCF); Stromal cell-derived factor 1 (SDF-1); and Vascular endothelial growth factor (VEGF).

Non-limiting examples of differentiation agents and combinations or “cocktails” of differentiation agents suitable for use in some embodiments described herein are provided in Table I. Table I also links the differentiation agents or combinations of differentiation agents to specific types of autologous biological tissue to be grown in a manner described herein using the differentiation agents.

TABLE I
Differentiation Agents.

| Differentiation Agents | Autologous Biological Tissue |
|---|---|
| Bone Morphogenic Protein (BMP) (e.g., BMP-2, BMP-6, | Bone |
| BMP-7) | |
| Epo | Bone |
| Transforming growth factor-beta (TGF-β) | Bone |
| Combination of VEGF + BMP | Bone |
| Combination of L-thyroxine + 20 mM β-glycerol phosphate + | Bone |
| dexamethansone + ascorbic acid | |
| Combination of L-glutamine + PEST + ascorbic acid + B- | Bone |
| glycerophosphate + dexamethasone | |
| Adenosine | Bone |
| Fibroblast growth factor-2 (FGF-2) | Vascular Tissue |
| BMP-4 | Vascular Tissue |
| Indian hedgehog protein (IHH) | Vascular Tissue |
| VEGF | Vascular Tissue |
| Combination of TGF-β + VEGF | Vascular Tissue |
| Combination of dexamethasone + ascorbic acid + L-proline + | Cartilage |
| ITS supplement + TGFβ-3 + BMP-2 | |
| Combination of linoleic acid + insulin + transferrin + | Cartilage |
| selenium + TGF-β1 + dexamethasone + ascorbic acid + | |
| PEST | |
| Combination of TGF-β3 + dexamethasone | Cartilage |
| Combination of insulin + transferrin + selenious acid + | Cartilage |
| dexamethasone + ascorbic acid-2 phosphate + sodium | |
| pyruvate + proline + TGF-β3 + BMP-6 | |
| Combination of dexamethasone + AsA + proline + TGF-β1 + | Cartilage |
| ITS⁺ premix + insulin + transferrin + selenious acid + | |
| linoleic acid | |
| Combination of IHH + BMP | Cartilage |
| Combination of TGF-β + BMP-6 | Cartilage |
| TGF-β3 | Tendon |
| Growth/differentation factors (GDFs) | Tendon |
| BMP-12 or GDF7 | Tendon |
| Combination of insulin + indomethacin + dexamethasone | Fat |
| Combination of PEST + insulin + dexamethasone + | Fat |
| isobutylmethylxanthine + indomethacin | |
| Combination of insulin + dexamethasone + indomethacin + | Fat |
| 3-isobutyl-1-methyl xanthine | |
| Combination of 3-isobutyl-1-methylxanthine + | Fat |
| hydrocortisone + indomethacin | |
| Combination of Epo + indomethacin | Fat |
| Glucose | Fat |
| Retinoic acid | Nerve Tissue |
| Combination of β-mercaptoethanol + epidermal growth | Nerve Tissue |
| factor + nerve growth factor + brain derived growth factor | |
| Combination of cyclic adenosine monophosphate + AsA | Nerve Tissue |
| Combination of FGF + SHH + BDNF + NGF + vitronectin + | Nerve Tissue |
| AsA + IBMX + forskolin + phorbol myristate acetate | |
| Combination of bFGF + retinoic acid + 2-mercaptoethanol | Nerve Tissue |
| B-catenin | Heart Valve Tissue |
| Combination of TGF-β + BMP | Heart Valve Tissue |
| VEGF | Heart Valve Tissue |
| NFATc1 | Heart Valve Tissue |
| ErbB | Heart Valve Tissue |
| NF1 | Heart Valve Tissue |
| Combination of Epo + Epidermalgrowth factor (EGF) | Kidney Tissue |
| Multiplication stimulation activities (MSA) | Kidney Tissue |
| Combination of TGF-β + EGF | Kidney Tissue |
| B-catenin | Kidney Tissue |
| Combination of EGF + glucose | Pancreas Tissue |
| Combination of Activin A + wortmannin | Pancreas Tissue |
| Combination of RA + FGF7 + NOGGIN | Pancreas Tissue |
| Combination of ITS + bFGF + nicotinamide + Exendin-4 + | Pancreas Tissue |
| BMP-4 | |
| Glucagon-like peptide-1 (GLP-1) | Pancreas Tissue |
| Combination of HGF + dexamethasone + ITS⁺ premix | Liver Tissue |
| Combination of OSM + dexamethasone + ITS⁺ premix | Liver Tissue |
| Activin A | Liver Tissue |
| Combination of FGF-4 + BMP-2 | Liver Tissue |
| Combination of dexamethasone + dimethyl sulfoxide | Liver Tissue |
| Combination of FGF-4 + hepatic growth factor | Liver Tissue |
| Combination of dexamethasone + insulin-transferrin- | Liver Tissue |
| selenium + oncostatin M | |

Further, biofactors such as chemokines and/or differentiation agents can be present in or on a scaffold in any amount not inconsistent with the objectives of the present disclosure. In some embodiments, for instance, a biofactor is present in a scaffold in an amount provided in Table II, where the amounts provided are per cubic centimeter (cm³) of total scaffold volume and where IU refers to International Units.

TABLE II
Biofactor Amounts (per cm³ scaffold).

```
  10 ng-10 mg
  10 ng-1 mg
  10 ng-100 μg
  50 ng-10 mg
  50 ng-1 mg
  50 ng-100 μg
  50 ng-10 μg
100 ng-10 mg
100 ng-1 mg
100 ng-100 μg
100 ng-10 μg
  1 μg-10 mg
  1 μg-1 mg
  1 μg-100 μg
  1 μg-50 μg
  1 μg-10 μg
  1-5000 IU
  1-3000 IU
  1-1000 IU
10-1000 IU
50-1000 IU
```

Further, in some cases, the amount or concentration of a biofactor or combination of biofactors described herein can be selected based on a desired cell differentiation and or tissue growth effect. For example, in some embodiments, the amount of a biofactor or combination of biofactors is chosen to selectively promote the growth of a desired autologous biological tissue at or on a surface of a scaffold, rather than merely within the general vicinity of the scaffold or within the body cavity (or other region) in which the scaffold is disposed. Non-limiting examples of some preferred amounts and types of biofactors for the promotion of autologous bone tissue growth are provided in Table III.

TABLE III
Biofactors and Amounts.

| | Amount |
|---|---|
| Biofactor | (per cm³ scaffold) |
| BMP-2 | 10-200 ng |
| BMP-7 | 20-400 ng |
| TGF-β2 | 100 ng-2 μg |
| VEGF | 100 ng-3 μg |
| Epo | 50-1000 IU |
| SDF-1α | 100 ng-10 μg |
| GCSF | 2-100 μg |
| HGF | 1-50 μg |
| RANTES | 1-50 μg |

Further, biofactors described herein can be disposed in or on a scaffold in any manner not inconsistent with the objectives of the present disclosure. For example, in some embodiments, one or more biofactors are disposed on or in a scaffold using protein microbubbles such as albumin microbubbles as a porogen. Such a technique is described, for instance, in Nair et al., “Novel polymeric scaffold using protein microbubbles as porogen and growth factor carriers,” Tissue Engineering 16, 23-32 (2010). In other instances, biofactors can be loaded into the scaffold by physical adsorption, chemical reaction, and/or the incorporation of particles or gels (such as hydrogels) comprising the biofactors. For example, in some instances, biofactors can be loaded into the scaffold through a chemical coupling scheme such as a carbodiimide coupling scheme.

Additionally, as described above, a scaffold described herein can release one or more biofactors in a sustained or prolonged manner. As described above, releasing biofactors in this manner can provide or create a localized gradient of biofactors proximate the scaffold and/or within the body cavity (or other region). Moreover, in some embodiments, such a release of biofactors can be achieved by disposing the biofactors on or in a porous scaffold described hereinabove, including in an amount or concentration described hereinabove. In some embodiments, a scaffold described herein can release biofactors from the scaffold for a time period of at least 1 week, at least 2 weeks, at least 3 weeks, at least 4 weeks, at least 5 weeks, or at least 6 weeks. In some instances, a scaffold described herein can release biofactors from the scaffold when disposed in a body cavity described herein for a time period of 1-10 weeks, 1-8 weeks, 1-6 weeks, 1-4 weeks, 2-10 weeks, 2-8 weeks, 2-6 weeks, 2-4 weeks, 4-10 weeks, 4-8 weeks, or 4-6 weeks.

Moreover, in some embodiments, a scaffold used in a method described herein does not include or contain seed cells, such as seed stem cells or progenitor cells. Instead, as described hereinabove, a scaffold described herein can induce growth of a desired autologous biological tissue within a body cavity (or other region) using only autologous cells recruited in vivo. Thus, a scaffold that is free or substantially free of multipotent cells or other seed cells can be fabricated and stored for longer periods of time prior to use than some other scaffolds, such as scaffolds that include living cells. As described herein, a scaffold that is “substantially free” of multipotent cells or other seed cells includes an insufficient number of cells to seed the growth of the autologous biological tissue on the scaffold without the recruitment of multipotent cells within the body cavity (or other region). A “seed” cell, as described herein, can be any cell that is used to seed the growth of autologous biological tissue. Such seed cells can, in some cases, be non-autologous. In other instances, such seed cells can be previously harvested and/or cultured cells obtained from the patient.

Turning again more particularly to specific steps of methods of growing autologous biological tissue, methods described herein can include promoting migration and/or differentiation of autologous multipotent cells. The autologous multipotent cells, in some embodiments, comprise stem cells or progenitor cells. For example, in some cases, the multipotent cells comprise mesenchymal stem cells (MSCs). Additionally, it is to be understood that “progenitor” cells can include multipotent cells that are more differentiated than stem cells and/or that can be differentiated into a specific “target” cell or cell type. In some embodiments described herein, progenitor cells comprise cells native to the body cavity of the method. For example, in some instances, the multipotent cells of a method described herein include peritoneal progenitor cells. Further, it is to be understood that “autologous” cells include cells that are produced by the patient. Moreover, as described hereinabove, such autologous cells can be recruited in vivo during the course of carrying out a method described herein, as opposed to being previously harvested, purified, cultured, and/or reinserted into the patient.

Additionally, in some embodiments, methods described herein further comprise differentiating deposited multipotent cells into a desired autologous biological tissue. As described hereinabove, it is to be understood that different specialized or differentiated autologous tissue (such as bone or vascular tissue) can be grown according to the differentiation agents used. Thus, in some cases, differentiation of autologous multipotent cells comprises osteogenic differentiation, such as differentiation of the multipotent cells into osteoblasts. Other types of differentiation are also contemplated, such as adipogenic differentiation, valvular differentiation, or beta cell differentiation. Further, differentiation of multipotent cells “into” autologous biological tissue can comprise one differentiation step or a plurality of differentiation steps toward the desired autologous biological tissue. Similarly, it is to be understood that “differentiated” or “specialized” tissue (or cells) can include tissue (or cells) that are more differentiated than the multipotent cells from which the tissue (or cells) are derived according to a method described herein.

Moreover, any autologous biological tissue not inconsistent with the objectives of the present disclosure can be grown by a method described herein. For example, in some cases, the desired autologous biological tissue comprises one or more of bone tissue, vascular tissue, cartilage tissue, tendon tissue, fat tissue, nerve tissue, heart valve tissue, kidney tissue, pancreas tissue, and liver tissue. Further, as described hereinabove, the autologous biological tissue, in some embodiments, is not native to the body cavity (or other region) in which the method is carried out.

Moreover, any body cavity not inconsistent with the objectives of the present disclosure may be used. In some instances, the body cavity comprises a soft tissue body cavity. For example, in some embodiments, the body cavity comprises the peritoneal cavity, the pleural cavity, the abdominal cavity, the thoracic cavity, or the pelvic cavity. In other cases, a body cavity of a method described herein can include a joint cavity such as a synovial cavity. Not intending to be bound by theory, it is believed that the use of a soft tissue body cavity described herein can permit access to the scaffold by a large number of multipotent cells, including multipotent cells that may migrate into the body cavity from another portion of the body and/or diffuse to the scaffold surface through the body cavity.

### II. Compositions

In another aspect, compositions are described herein which, in some embodiments, can be used to carry out a method of growing autologous biological tissue described hereinabove in Section I. In some cases, a composition described herein comprises a scaffold and one or more biofactors disposed in the scaffold. A composition described herein, in some embodiments, can also comprise cells and/or tissue disposed in or on the scaffold of the composition. For example, in some instances, a composition described herein can comprise differentiated or specialized tissue disposed in or on the scaffold, including differentiated or specialized tissue described hereinabove in Section I.

Turning now to specific components of compositions described herein, the scaffold of a composition can comprise any scaffold described hereinabove in Section I. For example, in some instances, the scaffold is a porous scaffold having a porosity and/or average pore size described in Section I. In some embodiments, for instance, the scaffold is a porous scaffold having an average pore size of 50-250 μm. Additionally, in some cases the scaffold of a composition described herein is biodegradable and/or biocompatible.

Similarly, the biofactors of a composition described herein can comprise any biofactor or combination of biofactors described hereinabove in Section I. For instances, in some cases, the biofactors comprise one or more chemokines that promote migration of autologous multipotent cells to a surface of the scaffold and/or one or more differentiation agents that promote differentiation of autologous multipotent cells into an autologous biological tissue. Further, in some embodiments, the biofactors comprise a combination of chemokines and differentiation agents. Any combination of chemokines and differentiation agents not inconsistent with the objectives of the present disclosure may be used. Moreover, the chemokines and/or differentiation agents can be selected based on a desired autologous biological tissue to be grown using the composition.

For example, in some cases, the biofactors disposed in a scaffold of a composition described herein comprise one or more of the biofactors of Table III. Thus, in some instances, a scaffold comprises one or more of BMP-2, present in the scaffold in an amount of 10-200 ng per cm³ scaffold; BMP-7, present in the scaffold in an amount of 20-400 ng per cm³ scaffold; TGF-β2, present in the scaffold in an amount of 100 ng to 2 μg per cm³ scaffold; VEGF, present in the scaffold in an amount of 100 ng to 3 μg per cm³ scaffold; Epo, present in the scaffold in an amount of 50-1000 International Units per cm³ scaffold; SDF-la, present in the scaffold in an amount of 100 ng to 10 μg per cm³ scaffold; GCSF, present in the scaffold in an amount of 2-100 μg per cm³ scaffold; HGF, present in the scaffold in an amount of 1-50 μg per cm³ scaffold; and RANTES, present in the scaffold in an amount of 1-50 μg per cm³ scaffold. As described hereinabove, such a composition can be used to promote the growth of autologous bone tissue in vivo, including in a body cavity or other region of a patient. Other combinations of biofactors can also be used in a scaffold of a composition described herein to promote the growth of other autologous biological tissues, such as one or more of vascular tissue, cartilage tissue, tendon tissue, fat tissue, nerve tissue, heart valve tissue, kidney tissue, pancreas tissue, and liver tissue.

Some embodiments described herein are further illustrated in the following non-limiting examples.

### Example 1

### Methods of Growing Autologous Biological Tissue

Methods of growing autologous biological tissue according to some embodiments described herein were carried out as follows. Specifically, a murine peritoneal implantation model was used to grow new autologous bone tissue in vivo.

### A. Ethics Statement

The animal use protocols used for this work were reviewed and approved by the Institutional Animal Care and Use Committee of the University of Texas at Arlington.

### B. Materials

Goat anti-mouse SCF and Nanog antibodies and rabbit anti-mouse antibodies were obtained from Santa Cruz Biotechnology Inc. (Santa Cruz, Calif., USA). Anti-mouse CD45, Sca-1, c-kit, CD34, FLK2-, CD3, B220, TER-119-, antibodies (rat anti mouse) to various stem cell markers CD105, CD29 and CD44, along with secondary antibody streptavidin PE/Cy5.5, and donkey anti-rat-APC were obtained from eBioscience (San Diego, Calif., USA). Rat anti mouse CD105 was obtained from Santa Cruz Biotechnology (Dallas, Tex., USA). Biotin conjugated lineage antibody cocktail was obtained from MiltenyiBiotec (MiltenyiBiotec Inc., Auburn, Calif., USA). Bone morphogenetic protein-2 (BMP-2) was obtained from R&D Systems (R&D Systems, Minneapolis, Minn., USA). PLLA was obtained from Medisorb 100L 1A (Lakeshore Biomaterials, AL, USA) with an inherent viscosity of 1.9 dL/g.

### C. Methods

### 1. Mouse Peritoneal Cell Collections

Balb/c mice (approximately 4-6 months old) were used. Mice were implanted with a polyurethane umbilical vessel catheter (2 cm in length, 5.0 FR, Sentry Medical Products, Lombard, Ill., USA) based on a modification of a previously published procedure. See Tang et al., “Fibrin(ogen) mediates acute inflammatory responses to biomaterials,” J. Exp. Med. 178: 2147-2156 (1993); Hu et al., “Molecular basis of biomaterial-mediated foreign body reactions,” Blood 98: 1231-1238 (2001). Briefly, the mice were sedated with isoflurane inhalation. Following sterilization with 70% ethanol, a small incision (approximately 5 mm) was made and two sections of catheter were implanted in the peritoneal cavities. The incisions were then closed with stainless steel wound clips. After implantation, the mice were euthanized at different times (0 h, 6 h, 12 h, 18 h, 1 d, 2 d, 4 d, 7 d, 10 d, 14 d) with carbon dioxide inhalation. The peritoneal cells were then recovered via peritoneal lavage with 5 mL of sterile saline twice. The isolated cells were then characterized by determining the expression of various cell markers and via cell differentiation studies.

### 2. Flow Cytometry Analyses and Cell Differentiation of Peritoneal Progenitor Cells

For flow cytometry analysis, RBC lysing buffer (Sigma Chemical Co., St Louis, Mo., USA) was used to remove red blood cells from each sample following the manufacturer's instructions as previously described. See Zheng et al., “Inhibitory receptors bind ANGPTLs and support blood stem cells and leukaemia development,” Nature 485: 656-660 (2012). The cell density was adjusted to 5×10⁶/mL and then stained with monoclonal antibodies including anti-mouse CD105, CD29, CD45, CD44, CD3, B220, Mac-1, and TER-119, or biotin conjugated lineage antibody cocktail (CD3, B220, CD11b, CD14, and Ter 119, MiltenyiBiotec). Streptavidin secondary antibody, Sca-1, c-kit, CD34, FLK2. Lin⁻Sca-1⁺Kit⁺CD34⁻FLK2− are widely used markers for long term hematopoietic stem cells (HSCs), while CD105⁺CD29⁺CD44⁺CD45⁻ is well recognized as the marker set for MSCs. Stained cells were analyzed on BD FACSCalibur (BD Bioscience, San Jose, USA) to determine the types and percentages of peritoneal cells. Osteogenic differentiation of peritoneal progenitor cells was performed on confluent cells in the presence of recombinant BMP-2 (R&D Systems, Minneapolis, Minn., USA) for 3 weeks as previously described. See Spinella-Jaegle et al., “Opposite effects of bone morphogenetic protein-2 and transforming growth factor-beta1 on osteoblast differentiation,” Bone 29: 323-330 (2001). Calcium-rich deposits by osteoblasts were then evaluated using Alizarin Red S staining. Adipogenic, neurogenic and myogenic differentiation of peritoneal progenitor cells was performed and analyzed according to a previously published procedure. See Nair et al., “Biomaterial implants mediate autologous stem cell recruitment in mice,” Acta Biomater 7: 3887-3895 (2011).

### 3. Induced Bone Formation in Peritoneal Cavity

Both decalcified bone collagen scaffolds and porous PLLA scaffolds were used for triggering bone formation in the peritoneal cavity. The decalcified bone scaffolds contained bone morphogenetic protein for inducing bone formation. It was estimated that there are 3 mg of BMP-2 per gram of demineralized dentin, and about 50-100 ng of a combination of BMPs per 25 mg of bovine bone matrix. Decalcified femur bone scaffolds (approximately 1.5 mm×1 mm×15 mm in size) were prepared according to published procedures. See, e.g., King et al., “Fractal dimension analysis of the cortical ribbon in mild Alzheimer's disease,” Neuroimage 53: 471-479 (2010); and Yaccoby et al., “Antibody-based inhibition of DKK1 suppresses tumor-induced bone resorption and multiple myeloma growth in vivo,” Blood 109: 2106-2111 (2007). PLLA scaffolds were fabricated using a salt leaching technique. See Thevenot et al., “Method to analyze three-dimensional cell distribution and infiltration in degradable scaffolds,” Tissue Eng Part C Methods 14: 319-331 (2008). More specifically, to stimulate localized osteogenic differentiation, ethanol sterilized PLLA scaffolds (5 mm×5 mm×5 mm in size with a pore size of 150 to 300 μm) were immersed in osteogenic differentiation solution (complete medium supplemented with 50 μg/mL ascorbic acid-2-phosphate, 10 nM dexamethasone, 7 mM β-glycerolphosphate, and 1 μg/ml BMP-2) overnight, then lyophilized prior to implantation. Studies indicated that such scaffolds exhibited in vivo biofactor release rates of approximately 5% (approximately 50 ng) per scaffold per day for a period of 2 weeks. For in vivo testing, the scaffolds, including untreated scaffolds as controls, were implanted in the peritoneal cavities of the mice. The implants were isolated at 16 hours, 2 weeks, 6 weeks, and 12 weeks for histological evaluation.

### 4. Histological Evaluation of Peritoneal Implants

All explants were frozen, cryosectioned, fixed, and used for various histological evaluations as described in the literature. See Nair et al., “Biomaterial implants mediate autologous stem cell recruitment in mice,” Acta Biomater 7: 3887-3895 (2011); and Shen et al., “Transplantation of mesenchymal stem cells from young donors delays aging in mice,” Sci Rep 1: 67 (2011). H&E stain was used for providing a general overview of tissue structures. Immunohistochemical analyses were performed for assessing the presence of stem and progenitor cell markers (including SCF and Nanog) and osteoblast markers (osteocalcin and alkaline phosphatase). Alkaline phosphatase activity (AP activity) was tested using a biochemical assay obtained from Sigma (St. Louis, Mo., USA). Calcium content change was tested by Alizarin Red S staining and von Kossa staining. All stained sections were observed under light microscopy (Leica DM LB) and the images analyzed using ImageJ.

### 5. Statistical Analyses

The extent of cell recruitment, stem cell marker expression, and mineralization were analyzed using one way ANOVA. PLLA scaffold mineralization at the end of 8 weeks was evaluated using Student's t-test. The statistical significance was determined at p<0.05.

### D. Results

### 1. Recruitment and Characterization of Peritoneal Fluid Cells

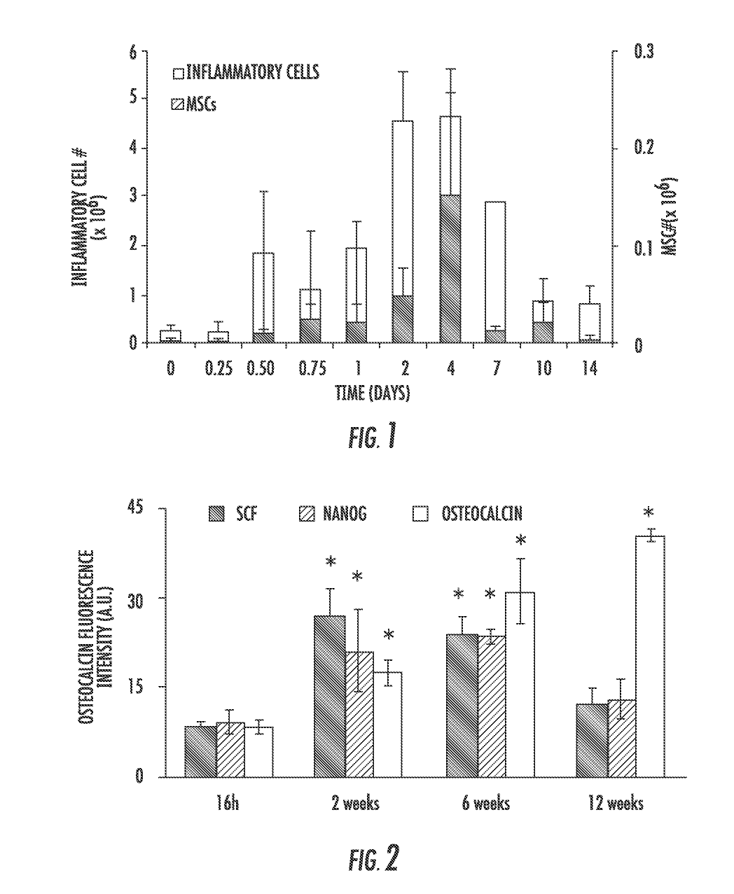

First, the cell populations that exist/arrive in the mice peritoneal cavity following introduction of an implant were investigated. Polyurethane umbilical vessel catheters (2 cm in length) were implanted into the mouse (n=5) peritoneal cavity to mimic the trauma and foreign body response caused by peritoneal dialysis procedures. After catheter implantation, peritoneal lavage fluid was collected at various time points up to day 14 from both catheter-implanted and control animals. After implantation for 2 days, a number of inflammatory cells such as T-cells (5.33±0.52%), B-cells (17.20±0.94%), myeloid cells (64.81±0.58%) and erythroid cells (4±0.41%) were predominantly observed. Interestingly, two unique sub-populations in the effluent cells that shared markers identical to those of MSCs (CD105⁺CD44⁺CD29⁺CD45⁻) and HSCs (Lin⁻Sca-1⁺Kit⁺CD34⁻FLK2⁻) were also observed in nearly all of the animals. These cells accounted for 0.29±0.04% and 0.03±0.01%, respectively. The observed cell population fractions are illustrated in FIG. 1. The peritoneal MSCs also expressed many progenitor cell markers, including CD8a, CD29, CD31, CD44, CD54, CD73, CD105, CD106, Stem Cell Factor (SCF), SH-3, Nanog, SSEA-3, and vimentin. They also stained negative for CD10, CD11b, CD11c, CD13, CD14, CD19, CD30, CD34, CD45, CD49e, CD90, CD95, CD117, CD166, Nestin, Neurofilament-N, STRO-1, TRA-1-81, or alpha-smooth muscle actin. Similar cells were also identified from the peritoneal effluents of human patients with End Stage Renal Disease (ESRD). To test the functionality and plasticity of these peritoneal progenitor cells, the multipotency of the cells was assessed by culturing them in vitro in the presence of various differentiation-inducting media. The undifferentiated cells exhibited fibroblast like morphology. The culture of these cells in specific differentiation media led to differentiation into osteogenic, adipogenic, neurogenic, and myogenic phenotypes. These results indicated that the implantation of catheters in the peritoneal cavity of adult mice prompted the migration into the area of multipotent MSCs and other progenitor cells that express markers similar to those expressed on bone marrow stem cells.

### 2. Calcification of Decalcified Bone Scaffold Implants

To determine the ability of the multipotent MSCs cells to form bone tissue in the peritoneal cavity, decalcified bone scaffolds (approximately 1.5 mm×1 mm×15 mm in size) were initially used. In this manner, the ability of intraperitoneal implant-recruited MSCs to (a) differentiate into bone forming cells and (b) produce mineralized bone tissue was evaluated. Since peritoneal MSCs were found to express SCF and Nanog, both markers were used to assess the extent of MSC recruitment following scaffold implantation. H&E staining of the bone scaffolds showed an increase in eosinic staining from 16 hours to 12 weeks along with high cell infiltration by 12 weeks. Shortly after implantation (16 hours), there were many recruited cells, including SCF⁺ and Nanog⁺ progenitor cells, and an increased number of cells on the surfaces of scaffold implants. Implant-associated osteoblast activity remained low as reflected by slight osteocalcin production on the tissue-scaffold interface of all the early evaluated time points.

After two weeks, the number of SCF⁺/Nanog⁺ progenitor cells was almost 2 times that found at 16 hours, indicating the recruitment and infiltration of progenitor cells into bone scaffolds. The number of SCF⁺/Nanog⁺ cells remained the same through week 6 and was substantially reduced by week 12. By week 2, osteocalcin expression increased almost 2 times compared to that at the end of 16 hours. By 6 weeks this increase was almost 3 times, and by week 12 osteocalcin expression was almost 4 times that at 16 hours. In addition, over the 6 to 12 week period, as the levels of osteocalcin increased, the expression of SCF and Nanog returned to the same levels as that at the end of 16 hours, indicating the differentiation of the SCF⁺/Nanog⁺ progenitor cells into osteocalcin⁺ osteoblasts. FIG. 2 illustrates observed expressions from 16 h to 12 weeks.

### 3. Mineralization in Decalcified Bone Implants

Mineralization of the decalcified bone scaffolds in the peritoneal cavity was determined based on alkaline phosphatase (AP) activity, Alizarin Red S staining, and von Kossa staining. The area fraction of the implants that were mineralized was determined using ImageJ (FIG. 3). It was found that AP activity increased at week 2 and that the tissue-associated AP activity remained stable from weeks 2 to 12. Alizarin Red S stain and von Kossa stain were also carried out to assess scaffold mineralization. There was no significant increase in calcium content from 16 hours to 2 weeks. However, scaffold mineralization dramatically increased during the period between week 6 and week 12, by more than 7 fold, compared to the period between 16 hours and 2 weeks.

### 4. Ossification of PLLA Scaffold Implants

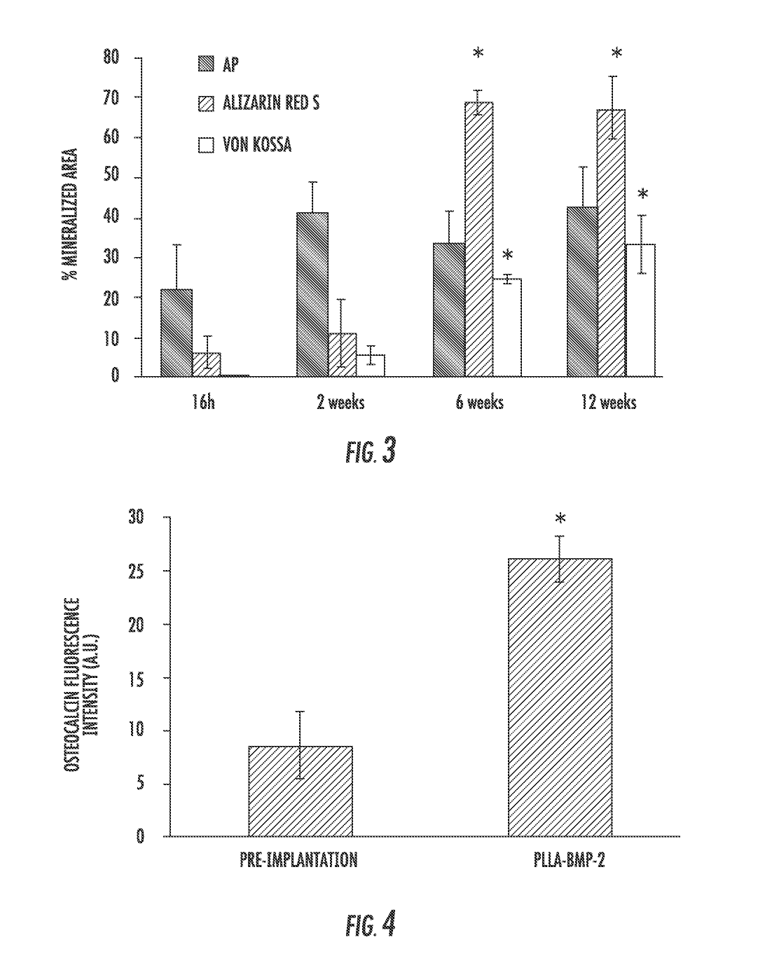

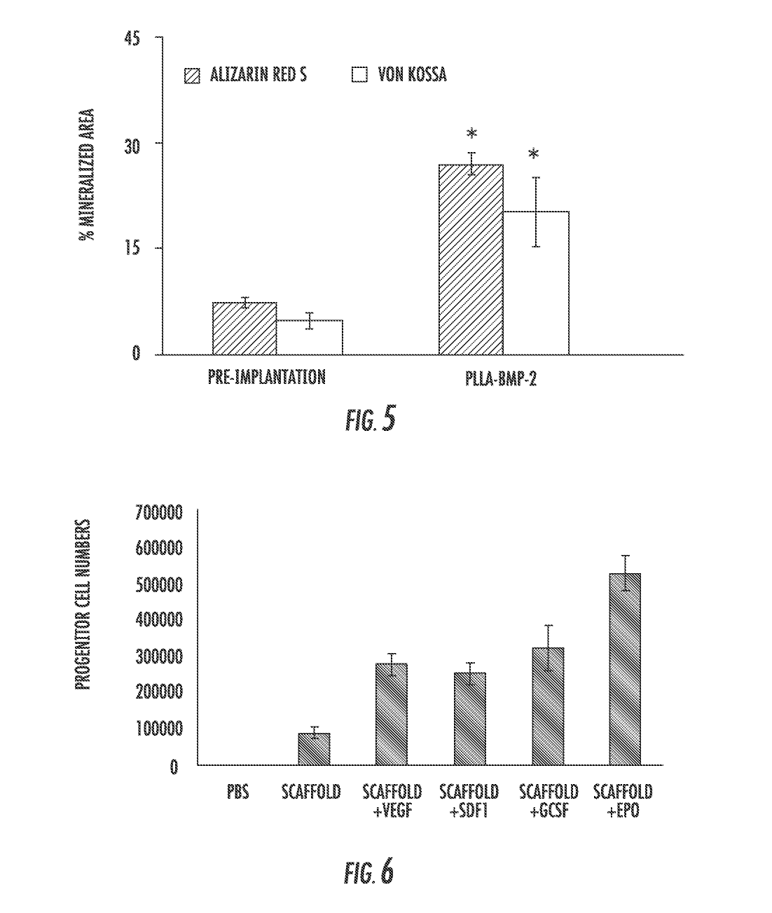

In addition to decalcified bone scaffolds, experiments were also carried out using poly-L-lactic acid (PLLA) polymer scaffolds that were loaded with BMP-2. Even without stem cell pre-seeding, PLLA scaffolds promoted the recruitment of MSCs (0.35±0.10%) by day 3. By week 8, substantial infiltration of cells into the scaffolds was observed (based on H&E staining). Many infiltrated cells expressed osteocalcin, indicating osteoblast activity. There was a nearly 5 fold increase in osteocalcin expression (FIG. 4). Signs of mineralization in the form of calcium and phosphate deposits were also observed within the scaffold. Quantification of markers of osteogenic activity showed a 5 fold increase in mineralization at the end of 8 weeks in PLLA scaffolds loaded with osteogenic differentiation agent BMP-2 (FIG. 5).

### Example 2

### Methods of Growing Autologous Biological Tissue

Methods of growing autologous biological tissue according to some embodiments described herein were carried out as follows. Specifically, a murine peritoneal implantation model was used to grow new autologous bone tissue in vivo. Unless stated otherwise herein, materials and methods used corresponded to those described hereinabove in Example 1.

### A. Multipotent Cell Recruitment

First, the “recruitment” of multipotent cells within the peritoneal cavity was evaluated by the localized release of chemokines from a scaffold. In particular, PLGA scaffolds were fabricated using the microbubble method described hereinabove. The scaffolds were then “loaded” with the following biofactors: vascular endothelial growth factor (VEGF), stromal derived factor-1 alpha (SDF-1α), erythropoietin (Epo), and granulocyte colony-stimulating factor (GCSF). The chemokine amounts or “dosages” (recombinant protein weight/scaffold volume) were as follows: VEGF (2 μg/cm³), SDF-la (10 ng/cm³), Epo (100 IU/cm³) and GCSF (5 μg/cm³). After maintaining the scaffolds in the peritoneal cavities for 24 hours, cells from the peritoneal fluid were recovered for flow cytometry analyses. The numbers of mesenchymal stem cells (CD105⁺/CD44⁺/CD29⁺/CD45⁻) were quantified. The results showed that animals (n=4 per biofactor or control group) with only phosphate buffered saline (PBS) injected (as a control) had only a small number of multipotent cells in the peritoneal cavities. Scaffolds alone (without biofactor, thus serving as another control) triggered weak multipotent cell recruitment. Scaffolds loaded with either VEGF, SDF-la, GCSF, or Epo triggered stronger multipotent cell recruitment than scaffold alone (FIG. 6).

### B. Differentiation of Multipotent Cells

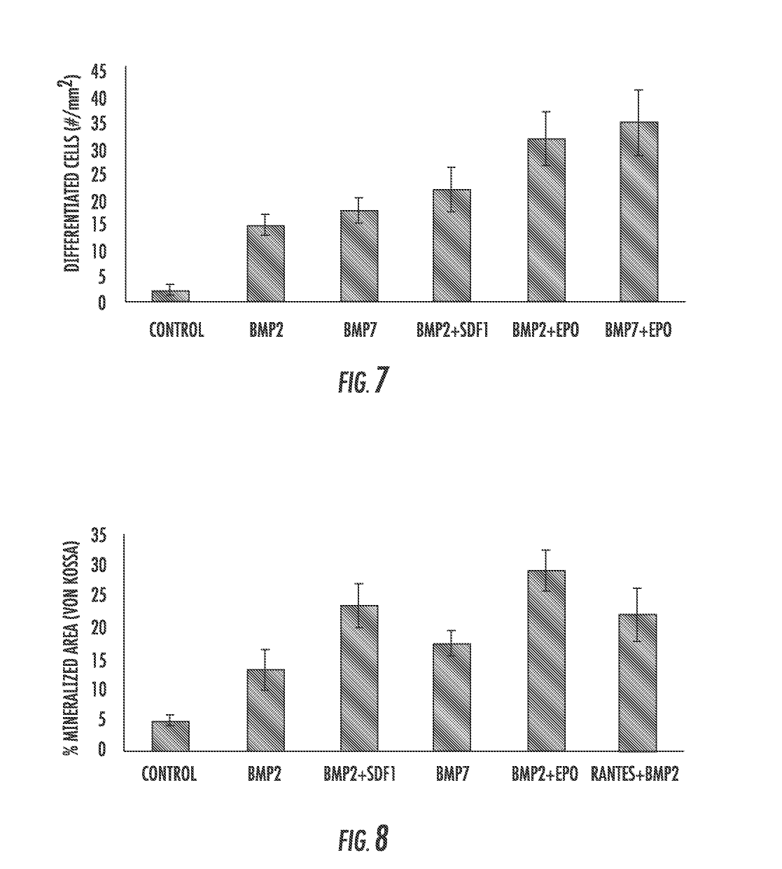

Next, the ability of various scaffolds to promote osteoblast differentiation was evaluated as follows. Six groups of PLGA scaffolds were fabricated: (1) control scaffolds containing no biofactors described herein; (2) scaffolds comprising BMP-2 (50 nanograms BMP-2/cm³); (3) scaffolds comprising BMP-7 (40 ng BMP-7/cm³); (4) scaffolds comprising a combination of BMP-2 and SDF-1α (50 ng BMP-2 and 10 ng SDF-1α per cm³); (5) scaffolds comprising a combination of BMP-2 and Epo (50 ng BMP-2 and 100 IU Epo per cm³); and (6) scaffolds comprising a combination of BMP-7 and Epo (40 ng BMP-7 and 100 IU Epo per cm³). The scaffolds were incubated with DMEM complete media (1 cm³ scaffold/5 mL DMEM w/10% fetal bovine serum) for 4 days. The scaffold-conditioned media were then added to mouse bone marrow stem cells (200 cells/mm²) for 21 days. The extent of cell differentiation was then determined using Alzarin Red staining for calcification deposition inside the cells. The BMP-2- and BMP-7-containing scaffold samples exhibited significant osteoblast differentiation. The scaffolds comprising a combination of biofactors exhibited even greater osteoblast differentiation. Results are illustrated in FIG. 7.

### C. Mineralization of Scaffolds

Porous scaffolds formed from PLGA and comprising one or more biofactors were disposed in the peritoneal cavities of mice. The mineralization of tissue in the scaffolds was then evaluated. Specifically, the scaffolds comprised one or more chemokines and/or one or more differentiation agents for bone tissue growth. The chemokine and differentiation agent amounts or dosages were as follows: BMP-2 (50 ng/cm³), BMP-7 (40 ng/cm³), SDF-1α (10 ng/cm³), Epo (100 IU/cm³), and RANTES (10 ng/cm³). As illustrated in FIG. 8, scaffolds comprising a combination of a chemokine and a differentiation agent (BMP-2+SDF1, BMP-2+Epo, and BMP-2+RANTES) produced more mineralized tissue than BMP2 or BMP7 alone. Control scaffolds (comprising no biofactors) induced minimal mineralized tissue formation.

### Example 3

### Methods of Growing Autologous Biological Tissue

Methods of growing autologous biological tissue according to some embodiments described herein were carried out as follows. Specifically, the methods described in Example 1 and Example 2 above were used to promote the growth of vascular tissue.

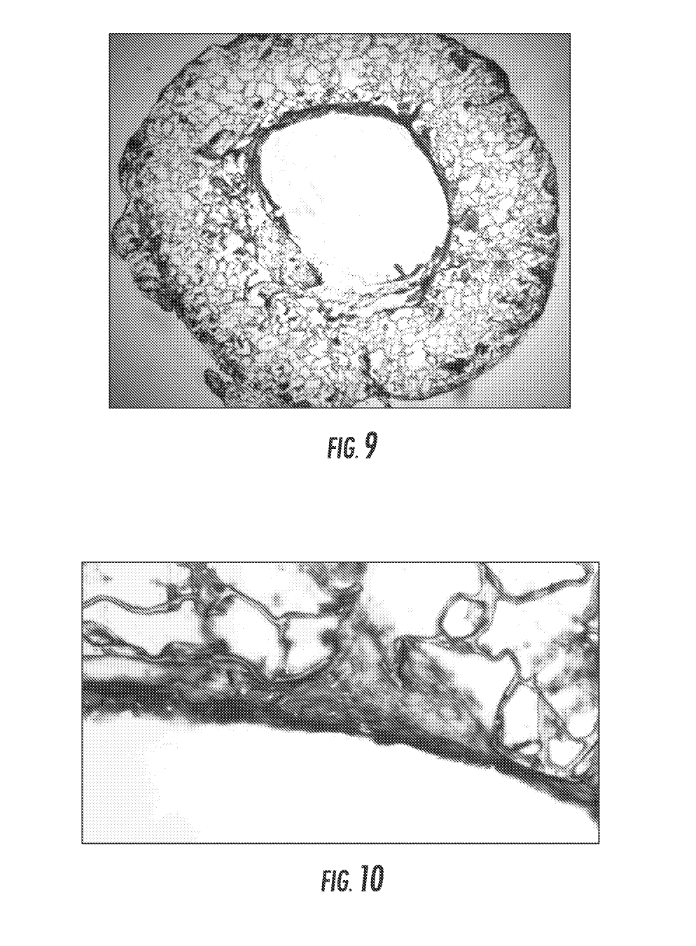

First, PLGA scaffolds were fabricated into tubular shapes (5 mm outer diameter and 2.5 mm inner diameter) using the microbubble method described hereinabove. The PLGA scaffolds were then loaded with VEGF (1 μg/cm³ scaffold) and implanted into the peritoneal cavities of mice. After implantation for 4 days, it was found that substantial endothelial progenitor cells were recruited to the peritoneal cavity (3.1±0.32% of total cells). The scaffold implants were analyzed after implantation for 14 days. At this time point, the scaffold implants were covered with endothelial progenitor cells. FIG. 9 illustrates a cross-section microscope image (at 100× magnification) of a scaffold following deposition of endothelial progenitor cells. As illustrated in FIG. 9, the inner lumen of the scaffold was covered with a layer of cells resembling endothelium. FIG. 10 illustrates an enlarged portion of the inner lumen illustrated in FIG. 9. As illustrated in FIG. 10, migration and proliferation of the vascular tissue inside the scaffold was observed.

Various embodiments of the present invention have been described in fulfillment of the various objectives of the invention. It should be recognized that these embodiments are merely illustrative of the principles of the present invention. Numerous modifications and adaptations thereof will be readily apparent to those skilled in the art without departing from the spirit and scope of the invention.

1. A composition comprising:
a scaffold; and
one or more biofactors disposed in the scaffold,
wherein the scaffold is a porous scaffold having an average pore size of 50-250 μm;
wherein the biofactors comprise one or more chemokines that promote migration of autologous multipotent cells to a surface of the scaffold and one or more differentiation agents that promote differentiation of autologous multipotent cells into an autologous biological tissue.

2. (canceled)

3. The composition of claim 1, wherein the biofactors comprise one or more of the following:
BMP2, present in the scaffold in an amount of 10-200 ng per cm³ scaffold;
BMP7, present in the scaffold in an amount of 20-400 ng per cm³ scaffold;
TGF-β2, present in the scaffold in an amount of 100 ng to 2 μg per cm³ scaffold;
VEGF, present in the scaffold in an amount of 100 ng to 3 μg per cm³ scaffold;
Epo, present in the scaffold in an amount of 50-1000 International Units per cm³ scaffold;
SDF-1α, present in the scaffold in an amount of 100 ng to 10 μg per cm³ scaffold;
GCSF, present in the scaffold in an amount of 2-100 μg per cm³ scaffold;
HGF, present in the scaffold in an amount of 1-50 μg per cm³ scaffold; and
RANTES, present in the scaffold in an amount of 1-50 μg per cm³ scaffold.

4. The composition of claim 1, wherein the scaffold does not include seed cells.

5. (canceled)

6. (canceled)

7. (canceled)

8. (canceled)

9. (canceled)

10. (canceled)

11. (canceled)

12. A method of growing autologous biological tissue comprising:
disposing a scaffold in a body cavity of a patient,
wherein the scaffold comprises one or more biofactors, and
wherein the biofactors comprise one or more chemokines that promote migration of autologous multipotent cells into the body cavity and one or more differentiation agents that promote differentiation of autologous multipotent cells into the autologous biological tissue.

13. (canceled)

14. The method of claim 12 further comprising releasing at least a portion of the biofactors into the body cavity from the scaffold.

15. The method of claim 12, wherein the multipotent cells are native to the body cavity.

16. The method of claim 12, wherein the multipotent cells are not native to the body cavity.

17. The method of claim 12, wherein the multipotent cells comprise stem cells or progenitor cells and wherein the scaffold does not include seed cells.

18. (canceled)

19. The method of claim 12 further comprising depositing the autologous multipotent cells onto an exterior surface and/or interior surface of the scaffold.

20. The method of claim 19 further comprising inducing differentiation of the autologous multipotent cells on the exterior surface and/or interior surface of the scaffold to provide differentiated autologous tissue.

21. The method of claim 20, wherein the differentiation is induced by the biofactors of the scaffold.

22. The method of claim 20 further comprising growing the autologous biological tissue from the differentiated autologous tissue on the exterior surface and/or interior surface of the scaffold.

23. The method of claim 12, wherein the autologous tissue is not native to the body cavity.

24. The method of claim 12, wherein the autologous biological tissue comprises one or more of bone tissue, vascular tissue, cartilage tissue, tendon tissue, fat tissue, nerve tissue, heart valve tissue, kidney tissue, pancreas tissue, and liver tissue.

25. The method of claim 24, wherein the autologous biological tissue comprises bone tissue.

26. (canceled)

27. The method of claim 12, wherein the body cavity comprises a soft tissue body cavity.

28. (canceled)

29. The method of claim 27, wherein the body cavity comprises the peritoneal cavity.

30. The method of claim 12 further comprising removing the autologous biological tissue from the body cavity of the patient and implanting the autologous biological tissue in a portion of the patient that differs from the body cavity of the patient.

31. (canceled)

32. The method of claim 30, wherein the implanted autologous biological tissue is attached to or disposed within the scaffold.

33. The method of claim 12, wherein the scaffold is formed from one or more of a poly-L-lactic acid (PLLA), poly-L-glycolic acid (PLGA), PLLA-PLGA copolymer or polymer blend, polycaprolactone, polydioxanone, poly-3-hydroxybutyrate, polytartronic acid, collagen, hyaluronic acid, and gelatin, and wherein the scaffold is a porous scaffold having an average pore size of 50-250 μm.

34. (canceled)
